(19) United States
(12) Patent Application Publication
(10) Pub. No.: US 2025/0011455 A1
(43) Pub. Date: Jan. 9, 2025

(54) ANTI-CD39 Antibody, Preparation Method Therefor And Use Thereof

(71) Applicant: Hangzhou Bangshun Pharmaceutical Co., Ltd., Yuhang District, Hangzhou, Zhejing (CN)

(72) Inventors: Zhaohui Li, Yuhang District, Hangzhou, Zhejing (CN); Min Wu, Yuhang District, Hangzhou, Zhejing (CN); Hedi Fang, Yuhang District, Hangzhou, Zhejing (CN); Qian Zhang, Yuhang District, Hangzhou, Zhejing (CN); Lixu Liu, Yuhang District, Hangzhou, Zhejing (CN); Lubin Lv, Yuhang District, Hangzhou, Zhejing (CN)

(73) Assignee: Hangzhou Bangshun Pharmaceutical Co., Ltd., Yuhang District, Hangzhou, Zhejing (CN)

(21) Appl. No.: 18/703,799

(22) PCT Filed: May 9, 2022
(86) PCT No.: PCT/CN2022/091703

§ 371 Date: Apr. 23, 2024

(30) Foreign Application Priority Data
May 12, 2021 (CN) 202110520014.6

(51) Int. Cl. C07K 16/28; A61K 39/00; A61P 35/00; G01N 33/574

(52) U.S. Cl. CPC C07K 16/2896; A61P 35/00; G01N 33/57484; A61K 2039/505; G01N 2333/70596

(57) ABSTRACT

Provided in the present invention is an anti-CD39 antibody, an antigen-binding fragment thereof, and pharmaceutical use thereof, and also provided are a chimeric antibody and a humanized antibody comprising CDR regions of the antibody, a pharmaceutical composition comprising the anti-CD39 antibody and the antigen-binding fragment thereof, and use of the antibody in preparing a medicament for treating diseases or disorders. The antibody of the present invention can specifically bind to CD39 and shows good effect in inhibiting tumor growth and good safety.

### TECHNICAL FIELD

The present invention relates to the field of biomedicine, and in particular to an anti-CD39 antibody or an antigen-binding fragment thereof.

### BACKGROUND

NTPDase 1 (ectonucleoside triphosphate diphosphohydrolase 1), also referred to as CD39, can hydrolyze extracellular adenosine triphosphate (ATP) and adenosine diphosphate (ADP) to generate adenosine monophosphate (AMP), which is further hydrolyzed by another enzyme, CD73 (exto-5′-nucleotidase), to generate adenosine, which binds to adenosine receptors and inhibits the responses of T cells and natural killer (NK) cells, thereby inhibiting the immune system. The generation of adenosine via CD73/CD39 is thought to be a major mechanism for the immunosuppressive function of regulatory T cells (Tregs).

Human CD39 is a 510-amino acid protein with 7 possible N-linked glycosylation sites, 11 cysteine residues, and 2 transmembrane domains. Structurally, it is characterized by 2 transmembrane domains, a small cytoplasmic domain containing NH2- and COOH-terminal segments, and a large extracellular hydrophobic domain consisting of 5 highly conserved domains which are called apyrase conserved regions (ACR) 1 to 5 and are critical for the catabolic activity of the enzyme. Although CD39 is typically anchored to the membrane by two transmembrane domains at two ends of the molecule, it has also recently been reported that a soluble catalytic activation form of CD39 has been shown to circulate in human and murine blood (Yegutkin et al., (2012) Federation of American Societies for Experimental Biology Journal (FASEB J.) 26 (9): 3875-3883).

CD39 is constitutively expressed in the spleen, thymus, lung, and placenta, and in these tissues, it is mainly associated with endothelial cells and immune cell populations such as regulatory T cells (Tregs) and natural killer (NK) cells. The expression of CD39 is increased in a plurality of solid tumors, e.g., colorectal cancer, head and neck cancer, pancreatic cancer (Kunzli et al., Am J Physiol, 2006, 292:223-230), bladder cancer, brain cancer, breast cancer, gastric cancer, hepatocellular carcinoma, lung cancer, non-small cell lung cancer (Li et al., Oncoimmunology, 2017, 6:6), chronic lymphocytic leukemia (Pulte et al., Clin Lymphoma Myeloma Leuk, 2011, 11 (4): 367-372) and lymphoma, melanoma (Dzhandzhugazyan et al., FEBS Letters, 1998, 430:227-230), ovarian cancer, and prostate cancer.

CD39, along with other enzymes, degrades ATP, ADP, and AMP to adenosine and adenosine binds to adenosine receptors and inhibits responses of T cells and natural killer (NK) cells, thereby inhibiting the immune system. CD39 regulators can activate effector T lymphocytes for tumor cell killing through the activation and clonal expansion of tumor-specific T cells, and thus CD39 regulators are potential therapies for these cancer types.

### SUMMARY

The present invention is intended to provide a novel anti-CD39 antibody or an antigen-binding fragment thereof, which can specifically bind to CD39, remarkably inhibit the enzymatic activity of CD39 for hydrolyzing ATP, and greatly reverse the inhibition of CD4+ T cell proliferation; the antibody or the antigen-binding fragment thereof shows good effect in inhibiting tumor growth, exhibits no toxic or side effects, and has good safety.

The present invention provides an anti-CD39 antibody or an antigen-binding fragment thereof, which comprises:
a heavy chain variable region comprising at least 1 HCDR as follows:
HCDR1, the amino acid sequence of the HCDR1 being set forth in SEQ ID NO: 1 or the HCDR1 comprising the amino acid sequence set forth in SEQ ID NO: 1;
HCDR2, the amino acid sequence of the HCDR2 being set forth in SEQ ID NO: 2 or the HCDR2 comprising the amino acid sequence set forth in SEQ ID NO: 2;
HCDR3, the amino acid sequence of the HCDR3 being set forth in SEQ ID NO: 3 or the HCDR3 comprising the amino acid sequence set forth in SEQ ID NO: 3; and/or
a light chain variable region comprising at least 1 LCDR as follows:
LCDR1, the amino acid sequence of the LCDR1 being set forth in SEQ ID NO: 4 or the LCDR1 comprising the amino acid sequence set forth in SEQ ID NO: 4;
LCDR2, the amino acid sequence of the LCDR2 being set forth in SEQ ID NO: 5 or the LCDR2 comprising the amino acid sequence set forth in SEQ ID NO: 5;
LCDR3, the amino acid sequence of the LCDR3 being set forth in SEQ ID NO: 6 or the LCDR3 comprising the amino acid sequence set forth in SEQ ID NO: 6.

In a preferred embodiment of the present invention, provided is an anti-CD39 antibody or an antigen-binding fragment thereof, wherein the antibody heavy chain variable region comprises the HCDR1, the HCDR2, and the HCDR3 having the amino acid sequences set forth in SEQ ID NO: 1, SEQ ID NO: 2, and SEQ ID NO: 3, respectively.

In a preferred embodiment of the present invention, provided is an anti-CD39 antibody or an antigen-binding fragment thereof, wherein the antibody light chain variable region comprises the LCDR1, the LCDR2, and the LCDR3 having the amino acid sequences set forth in SEQ ID NO: 4, SEQ ID NO: 5, and SEQ ID NO: 6, respectively.

In a preferred embodiment of the present invention, provided is an anti-CD39 antibody or an antigen-binding fragment thereof, wherein the antibody heavy chain variable region comprises the HCDR1, the HCDR2, and the HCDR3 having the amino acid sequences set forth in SEQ ID NO: 1, SEQ ID NO: 2, and SEQ ID NO: 3, respectively; and/or the antibody light chain variable region comprises the LCDR1, the LCDR2, and the LCDR3 having the amino acid sequences set forth in SEQ ID NO: 4, SEQ ID NO: 5, and SEQ ID NO: 6, respectively.

In a preferred embodiment of the present invention, provided is the anti-CD39 antibody or the antigen-binding fragment thereof according to the present invention, which further comprises a heavy chain FR region of a murine IgG1, IgG2, IgG3 or IgG4 or a variant thereof and/or further comprises a light chain FR region of a murine κ or λ chain or a variant thereof.

In a preferred embodiment of the present invention, provided is the anti-CD39 antibody or the antigen-binding fragment thereof according to the present invention, wherein the amino acid sequence of the heavy chain variable region is set forth in SEQ ID NO: 7 or has at least 90%, at least 95%, at least 96%, at least 97%, at least 98%, or at least 99% sequence identity to SEQ ID NO: 7, and/or the amino acid sequence of the light chain variable region is set forth in SEQ ID NO: 8 or has at least 90%, at least 95%, at least 96%, at least 97%, at least 98%, or at least 99% sequence identity to SEQ ID NO: 8.

In a preferred embodiment of the present invention, provided is the anti-CD39 antibody or the antigen-binding fragment thereof according to the present invention, wherein the antibody is a murine antibody or a fragment thereof.

In a preferred embodiment of the present invention, provided is the murine antibody or the fragment thereof according to the present invention, which further comprises a heavy chain constant region of a murine IgG1, IgG2, IgG3 or IgG4 or a variant thereof.

In a preferred embodiment of the present invention, provided is the murine antibody or the fragment thereof according to the present invention, which further comprises a light chain constant region of a murine κ or λ chain or a variant thereof.

In a preferred embodiment of the present invention, provided is the anti-CD39 antibody or the antigen-binding fragment thereof according to the present invention, wherein the antibody is a chimeric antibody or a fragment thereof.

In a preferred embodiment of the present invention, provided is the anti-CD39 chimeric antibody or the fragment thereof according to the present invention, which further comprises a heavy chain constant region of a human IgG1, IgG2, IgG3 or IgG4 or a variant thereof, preferably a heavy chain constant region of a human IgG4 or a variant thereof.

In a preferred embodiment of the present invention, provided is the anti-CD39 chimeric antibody or the fragment thereof according to the present invention, which further comprises a light chain constant region of a human κ or λ chain or a variant thereof, preferably a light chain constant region of human κ or a variant thereof.

In a preferred embodiment of the present invention, provided is the anti-CD39 chimeric antibody or the fragment thereof according to the present invention, wherein the heavy chain amino acid sequence of the antibody is set forth in SEQ ID NO: 9 or has at least 90%, at least 95%, at least 96%, at least 97%, at least 98%, or at least 99% sequence identity to SEQ ID NO: 9, and the light chain amino acid sequence of the antibody is set forth in SEQ ID NO: 10 or has at least 90%, at least 95%, at least 96%, at least 97%, at least 98%, or at least 99% sequence identity to SEQ ID NO: 10.

In a preferred embodiment of the present invention, provided is the anti-CD39 antibody or the antigen-binding fragment thereof according to the present invention, wherein the antibody is a humanized antibody or a fragment thereof.

In a preferred embodiment of the present invention, provided is the anti-CD39 humanized antibody or the fragment thereof according to the present invention, wherein the heavy chain variable region further comprises a heavy chain FR region of a human IgG1, IgG2, IgG3 or IgG4 or a variant thereof, preferably a FR region of human germline heavy chain IGHV1-2*02 or a variant thereof.

In a preferred embodiment of the present invention, provided is the anti-CD39 humanized antibody or the fragment thereof according to the present invention, wherein the light chain variable region further comprises a light chain FR region of a human κ or λ chain or a variant thereof, preferably a FR region of human germline light chain IGKV1-33*01 or a variant thereof. In a preferred embodiment of the present invention, provided is the anti-CD39 humanized antibody or the fragment thereof according to the present invention, wherein the amino acid sequence of the heavy chain variable region is set forth in SEQ ID NO: 11, 12, 13, 14, or 15 or has at least 90%, at least 95%, at least 96%, at least 97%, at least 98%, or at least 99% sequence identity to SEQ ID NO: 11, 12, 13, 14, or 15.

In a preferred embodiment of the present invention, provided is the anti-CD39 humanized antibody or the fragment thereof according to the present invention, wherein the amino acid sequence of the light chain variable region is set forth in SEQ ID NO: 16 or 17 or has at least 90%, at least 95%, at least 96%, at least 97%, at least 98%, or at least 99% sequence identity to SEQ ID NO: 16 or 17.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 11 or has at least 90%, at least 95%, at least 96%, at least 97%, at least 98%, or at least 99% sequence identity to SEQ ID NO: 11, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 12 or has at least 90%, at least 95%, at least 96%, at least 97%, at least 98%, or at least 99% sequence identity to SEQ ID NO: 12, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 13 or has at least 90%, at least 95%, at least 96%, at least 97%, at least 98%, or at least 99% sequence identity to SEQ ID NO: 13, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 14 or has at least 90%, at least 95%, at least 96%, at least 97%, at least 98%, or at least 99% sequence identity to SEQ ID NO: 14, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 15 or has at least 90%, at least 95%, at least 96%, at least 97%, at least 98%, or at least 99% sequence identity to SEQ ID NO: 15, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 11 or has at least 90%, at least 95%, at least 96%, at least 97%, at least 98%, or at least 99% sequence identity to SEQ ID NO: 11, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 12 or has at least 90%, at least 95%, at least 96%, at least 97%, at least 98%, or at least 99% sequence identity to SEQ ID NO: 12, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 13 or has at least 90%, at least 95%, at least 96%, at least 97%, at least 98%, or at least 99% sequence identity to SEQ ID NO: 13, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 14 or has at least 90%, at least 95%, at least 96%, at least 97%, at least 98%, or at least 99% sequence identity to SEQ ID NO: 14, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17.

In a preferred embodiment of the present invention, provided is the anti-CD39 humanized antibody or the fragment thereof according to the present invention, wherein the amino acid sequence of the heavy chain variable region of the anti-CD39 antibody or the antigen-binding fragment thereof is set forth in SEQ ID NO: 15 or has at least 90%, at least 95%, at least 96%, at least 97%, at least 98%, or at least 99% sequence identity to SEQ ID NO: 15, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17.

In a preferred embodiment of the present invention, provided is the anti-CD39 humanized antibody or the fragment thereof according to the present invention, which further comprises a heavy chain constant region of a human IgG1, IgG2, IgG3 or IgG4 or a variant thereof, preferably a heavy chain constant region of a human IgG4 or a variant thereof; more preferably, the amino acid sequence of the heavy chain constant region of the human IgG4 is set forth in SEQ ID NO: 18 or has at least 90%, at least 95%, at least 96%, at least 97%, at least 98%, or at least 99% sequence identity to SEQ ID NO: 18.

In a preferred embodiment of the present invention, provided is the anti-CD39 humanized antibody or the fragment thereof according to the present invention, which further comprises a light chain constant region of a human κ or λ chain or a variant thereof, preferably a light chain constant region of a human κ or a variant thereof; more preferably the amino acid sequence of the human κ light chain constant region is set forth in SEQ ID NO: 19 or has at least 90%, at least 95%, at least 96%, at least 97%, at least 98%, or at least 99% sequence identity to SEQ ID NO: 19.

Those skilled in the art can easily understand the complete sequences of the heavy chain and the light chain of the antibody according to the amino acid sequences of the variable regions and the constant regions of the heavy chain and the light chain of the antibody, and thus can obtain the complete information of the sequences of the antibody.

In a preferred embodiment of the present invention, provided is the anti-CD39 antibody or the fragment thereof according to the present invention, wherein the light chain variant of the anti-CD39 antibody or the fragment thereof preferably has 0-10 amino acid changes in the light chain variable region.

In a preferred embodiment of the present invention, provided is the anti-CD39 antibody or the fragment thereof according to the present invention, wherein the heavy chain variant of the anti-CD39 antibody or the fragment thereof preferably has 0-10 amino acid changes in the heavy chain variable region.

In a preferred embodiment of the present invention, provided is the anti-CD39 antibody or the antigen-binding fragment thereof according to the present invention, wherein the antigen-binding fragment is selected from Fab, Fv, scFv, Fab′, or F(ab′)².

The present invention further provides a biomaterial, which may be:
(1) a DNA molecule encoding the anti-CD39 antibody or the antigen-binding fragment thereof described above, wherein the DNA molecule may encode the heavy and light chain portions of the antibody, and those skilled in the art can deduce the DNA sequence based on the amino acid sequence of the antibody or the antigen-binding fragment thereof and set appropriate expression elements for it, enabling the DNA molecule to express the antibody or the antigen-binding fragment thereof of the present invention;
(2) an expression vector containing the DNA molecule described above;
(3) a host cell containing the DNA molecule or the expression vector described above, or a culture such as a culture solution or a bacterial suspension obtained by culturing the host cell.

In a preferred embodiment of the present invention, provided is the host cell according to the present invention, wherein the host cell is preferably a human embryonic kidney 293 cell or a Chinese hamster ovary cell.

The present invention further provides a method for producing an anti-CD39 antibody or an antigen-binding fragment thereof, which comprises culturing the host cell described above; further, the method comprises isolating an antibody from the obtained culture and purifying the antibody.

The present invention further provides a pharmaceutical composition, which comprises the anti-CD39 antibody or the antigen-binding fragment thereof according to the present invention and a pharmaceutically acceptable excipient, diluent, or carrier.

The present invention further provides a detection or diagnostic kit, which comprises the anti-CD39 antibody or the antigen-binding fragment thereof according to the present invention and is used in the detection, diagnosis, and prognosis of CD39 or a CD39-mediated disease or disorder.

The present invention further provides use of the anti-CD39 antibody or the antigen-binding fragment thereof or the biomaterial (such as the DNA molecule, the expression vector, and the host cell and the culture thereof) according to the present invention in preparing a medicament for treating or preventing a CD39-mediated disease or disorder.

The present invention further provides use of the anti-CD39 antibody or the antigen-binding fragment thereof, the biomaterial (such as the DNA molecule, the expression vector, and the host cell and the culture thereof), the pharmaceutical composition, and the kit according to the present invention in the detection, diagnosis, and prognosis of CD39 or a CD39-mediated disease or disorder.

The present invention further provides a method for treating and preventing a CD39-mediated disease or disorder, which comprises administering to a patient in need thereof a therapeutically effective amount of the anti-CD39 antibody or the antigen-binding fragment thereof, the biomaterial (e.g., the DNA molecule, the expression vector, and the host cell and the culture thereof), or the pharmaceutical composition according to the present invention.

The CD39-mediated disease or disorder described herein is a tumor expressing CD39, more preferably multiple myeloma, colorectal cancer, head and neck cancer, pancreatic cancer, bladder cancer, brain cancer, breast cancer, gastric cancer, hepatocellular cancer, lung cancer, leukemia, lymphoma, melanoma, ovarian cancer, prostate cancer, pituitary cancer, esophageal cancer, soft tissue sarcoma, peritoneal cancer, glioblastoma, cervical cancer, uterine cancer, salivary gland cancer, kidney cancer, vulvar cancer, thyroid cancer, testicular cancer, cholangiocarcinoma, or gallbladder cancer.

### BRIEF DESCRIPTION OF THE DRAWINGS

FIG. 1 shows the binding activity of the chimeric antibody F03 and control antibody for human CD39 on the cell surface.

FIG. 2 shows the inhibition of the enzymatic activity of CD39 on the cell surface for hydrolyzing ATP by the chimeric antibody F03 and control antibody.

FIG. 3 shows the reversal of the inhibition of T cell proliferation by the humanized antibodies.

FIG. 4 shows the effect of the humanized antibody in resisting multiple myeloma in vivo.

FIG. 5 shows the effect of the humanized antibody in resisting melanoma in vivo.

### DETAILED DESCRIPTION

### Terminologies and Definitions

In order to facilitate the understanding of the present invention, certain technical and scientific terms are specifically defined below. Unless otherwise specifically defined herein, all other technical and scientific terms used herein have the meanings generally understood by those of ordinary skill in the art to which the present invention belongs.

The three-letter and single-letter codes for amino acids used in the present invention are as described in J. biol. chem, 243, p 3558 (1968).

The term “antibody” described herein invention refers to an immunoglobulin, which is of a tetrapeptide chain structure formed by connection of two identical heavy chains and two identical light chains by interchain disulfide bonds. Accordingly, immunoglobulins can be divided into five classes, also called isotypes of immunoglobulins, namely IgM, IgD, IgG, IgA, and IgE, with their corresponding heavy chains being u chain, 8 chain, y chain, a chain, and & chain, respectively. Ig of the same class can be divided into different subclasses according to differences in the amino acid composition of the hinge regions and the number and positions of disulfide bonds of the heavy chains; for example, IgG can be divided into IgG1, IgG2, IgG3, and IgG4. Light chains are divided into κ or λ chains according to differences in the constant regions. Each of the five classes of Ig may have a κ chain or λ chain.

In the present invention, the antibody light chain described herein may further comprise a light chain constant region comprising a light chain constant region of a human or murine κ or λ chain or a variant thereof.

In the present invention, the antibody heavy chain described herein may further comprise a heavy chain constant region comprising a heavy chain constant region of human or murine IgG1, IgG2, IgG3 or IgG4 or a variant thereof.

In the antibody heavy and light chains, the sequences of about 110 amino acids near the N-terminus vary considerably and thus are referred to as variable regions (V regions); the remaining amino acid sequences near the C-terminus are relatively stable and thus are referred to as constant regions (C regions). The variable regions comprise 3 hypervariable regions (HVRs) and 4 framework regions (FRs) with relatively conservative sequences. The 3 hypervariable regions determine the specificity of the antibody and thus are also known as complementarity determining regions (CDRs). Each light chain variable region (LCVR) or heavy chain variable region (HCVR) consists of 3 CDRs and 4 FRs arranged from the amino-terminus to the carboxy-terminus in the following order: FR1, CDR1, FR2, CDR2, FR3, CDR3 and FR4. The 3 CDRs of the light chain refer to LCDR1, LCDR2, and LCDR3, and the 3 CDRs of the heavy chain refer to HCDR1, HCDR2, and HCDR3. The CDR amino acid residues of the LCVRs and HCVRs of the antibodies or antigen-binding fragments described herein correspond with known Kabat numbering scheme in terms of number and positions.

The term “murine antibody” used herein refers to an anti-CD39 monoclonal antibody prepared according to the knowledge and skill in the art. During the preparation, a test subject (mouse) is injected with the CD39 antigen, and then antibodies expressing the desired sequences or functional properties are isolated. In a preferred embodiment of the present invention, the murine anti-CD39 antibody or the antigen-binding fragment thereof may further comprise a light chain constant region of a murine κ or λ chain or a variant thereof, or further comprise a heavy chain constant region of a murine IgG1, IgG2, IgG3 or IgG4 or a variant thereof.

The term “chimeric antibody” refers to an antibody obtained by fusing a variable region of a murine antibody and a constant region of a human antibody, which can reduce an immune response induced by the murine antibody. A chimeric antibody is established by firstly establishing a hybridoma secreting a murine specific monoclonal antibody, then cloning a variable region gene from the mouse hybridoma cells, cloning a constant region gene of a human antibody as required, linking the mouse variable region gene and the human antibody constant region gene to form a chimeric gene, inserting the chimeric gene into a vector, and finally expressing the chimeric antibody molecule in a eukaryotic industrial system or prokaryotic industrial system.

In a preferred embodiment of the present invention, the heavy chain variable region of the anti-CD39 chimeric antibody further comprises a heavy chain FR region of a murine IgG1, IgG2, IgG3 or IgG4 or a variant thereof; preferably, the sequence of the antibody heavy chain variable region is set forth in SEQ ID NO: 7. The light chain variable region of the anti-CD39 chimeric antibody further comprises a light chain FR region of a murine κ or λ chain or a variant thereof; preferably, the sequence of the antibody light chain variable region is set forth in SEQ ID NO: 8. The constant region of the chimeric antibody may be selected from a heavy chain constant region of a human IgG1, IgG2, IgG3 or IgG4 or a variant thereof; preferably, the chimeric antibody comprises a heavy chain constant region of a human IgG4. The light chain constant region of the chimeric antibody may be selected from a light chain constant region of a human κ or λ chain or a variant thereof; preferably, the chimeric antibody comprises a light chain constant region of a human κ.

The term “humanized antibody”, also known as a CDR-grafted antibody, refers to grafting non-human (e.g., mouse) CDR sequences into the framework of variable regions of a human antibody without significantly affecting antigen binding properties. Humanized antibodies can overcome the disadvantage of strong immune response induced by a chimeric antibody because of carrying a large amount of mouse protein components. Such framework sequences can be obtained from public DNA databases or published references that include germline antibody gene sequences. For example, germline DNA sequences of genes of the human heavy and light chain variable regions can be found in the “VBase” human germline sequence database (available at the Internet address www.mrccpe.com.ac.uk/vbase), as well as in Kabat, E. A. et al., 1991 Sequences of Proteins of Immunological Interest, 5th edition. To avoid the decrease in activity caused by the decrease in immunogenicity, the variable regions of a human antibody can be subjected to minimum reverse mutation to maintain activity.

The “antigen-binding fragment” described herein refers to a Fab fragment, a Fab′ fragment, a F(ab′)² fragment, a Fv fragment, and a scFv fragment having binding activity for human CD39 antigen and comprises one or more CDRs in SEQ ID NO: 1 to SEQ ID NO: 6 described herein. The Fv fragment comprises the heavy chain variable region and the light chain variable region of the antibody but does not comprise the constant region, and has the smallest antibody fragment of the entire antigen-binding sites. Generally, the Fv antibody also comprises a polypeptide linker between the VH and VL domains and is capable of forming the structure required for antigen binding. Two antibody variable regions can also be linked, by using different linkers, to form a single polypeptide chain, known as a single chain antibody or a single chain Fv (scFv).

The Fab fragment refers to a monovalent fragment consisting of VL, VH, CL, and CHI domains. The F(ab′)² refers to a bivalent fragment formed by two Fab′ fragments linked by a disulfide bond at the hinge region.

Methods for producing and purifying antibodies and antigen-binding fragments are well known in the art and can be found in, for example, “Antibodies: A Laboratory Manual”, Cold Spring Harbor Press (chapters 5-8 and 15). For example, mice can be immunized with human CD39 or a fragment thereof, and the obtained antibodies can be renatured and purified, and amino acid sequencing can be performed by using conventional methods. Likewise, antigen-binding fragments can be prepared by conventional methods.

The monoclonal antibody or mAb described herein refers to an antibody obtained from a single clonal cell strain, which is not limited to eukaryotic, prokaryotic, or phage clonal cell strains. The monoclonal antibody or the antigen-binding fragment can be obtained by, for example, hybridoma technology, recombinant technology, phage display technology, synthetic technology (e.g., CDR-grafting), or other technologies known in the art.

“Affinity” or “binding property” refers to the sum of strength of non-covalent interactions between a single binding site of a molecule (e.g., an antibody) and its binding target (e.g., an antigen). As used herein, unless otherwise indicated, “binding affinity” refers to the inherent binding affinity that reflects the 1:1 interaction between members of a binding pair (e.g., an antibody and an antigen). The affinity of a molecule X for its binding target Y is generally expressed by the dissociation constant (KD). The affinity can be determined by conventional methods known in the art, including those described herein, e.g., using surface plasmon resonance (SPR) technology, such as an instrument.

“Specific binding or specifically binds to”, “specific for”, “selectively binds”, and “selective for” a particular antigen (e.g., a polypeptide target) or an epitope thereof refers to binding that is measurably different from non-specific or non-selective interaction. Specific binding can be determined, for example, by determining the binding of a determined molecule compared with that of a control molecule. Specific binding can also be determined by competition with a control molecule that is similar to the target, such as an excess of unlabeled target. The term “kd” (sec−1) used herein refers to the dissociation rate constant for a particular antibody-antigen interaction. This value is also referred to as the k dissociation value. The term “ka” (M−1×sec−1) used herein refers to the association rate constant for a particular antibody-antigen interaction. This value is also referred to as the k association value. The term “KD” (M) used herein refers to the dissociation equilibrium constant for a particular antibody-antigen interaction. KD=kd/ka.

“Give” and “treat”, when applied to animals, humans, experimental subjects, cells, tissues, organs, or biological fluids, refer to contacting an exogenous drug, therapeutic agent, diagnostic agent, or composition with the animals, humans, subjects, cells, tissues, organs, or biological fluids. “Give” and “treat” can refer to, for example, therapeutic, pharmacokinetic, diagnostic, research, and experimental methods. The treatment of cells comprises contacting the reagent with the cells and contacting the reagent with fluid, where the fluid is in contact with the cells. “Give” and “treat” also refer to treating, e.g., cells by reagents, diagnosis, binding compositions or by another cell in vitro and ex vivo. “Treat”, when applied to humans, veterinary or research subjects, refers to therapeutic treatment, preventive or prophylactic measures, and research and diagnostic applications.

“Treating” or “treatment” refers to administering a therapeutic agent, such as a composition comprising any one of the anti-CD39 antibodies or the antigen-binding fragments thereof of the present invention, cither internally or externally to a patient with one or more symptoms of a disease on which the therapeutic agent is known to have a therapeutic effect. Typically, the therapeutic agent is administered in an amount effective in alleviating one or more symptoms of the disease in the patient or population being treated, whether by inducing regression of such symptoms or inhibiting the progression of such symptoms to any clinical degree. The amount of the therapeutic agent effective in alleviating any particular symptom of the disease (also known as a “therapeutically effective amount”) may vary depending on a variety of factors, such as the disease state, age, and weight of the patient, and the ability of the drug to produce a desired therapeutic effect in the patient. Whether a symptom of a disease has been alleviated can be assessed by any clinical testing methods generally used by physicians or other health care professionals to assess the severity or progression of the symptom, for example, by a statistical test method known in the art, such as Student t-test, chi-square test, U-test by Mann and Whitney, Kruskal-Wallis test (H-test), Jonckheere-Terpstra test, and Wilcoxon test.

“Conservative modification” or “conservative replacement or substitution” refers to replacement of amino acids in a protein with other amino acids having similar characteristics (e.g., charge, side-chain size, hydrophobicity/hydrophilicity, or backbone conformation and rigidity), without changing the biological activity of the protein. Those skilled in the art know that, generally speaking, a single amino acid replacement in a non-essential region of a polypeptide does not substantially change the biological activity (see, e.g., Watson (1987) Molecular Biology of the Gene, The Benjamin/Cummings Pub. Co., p 224, (4th edition)). In addition, the replacement of amino acids with similar structure or function is unlikely to disrupt the biological activity. In the present invention, the variant of the antibody light chain or heavy chain is “conservative modification” or “conservative substitution replacement or substitution” of 0-10 amino acids in the light chain or heavy chain, and those skilled in the art can expect that the variant has substantially the same activity as that before the modification or substitution. In addition, the variant of the light chain or heavy chain of the antibody described herein further comprises the result of a back mutation, i.e., back mutation of amino acids of a certain humanization template of the humanized antibody FR region to the murine amino acids of the corresponding positions. Those skilled in the art can expect that the variant has comparable or better activity than the humanized antibody before back mutation and the murine antibody containing the same CDRs.

“Effective amount” comprises an amount sufficient to ameliorate or prevent a medical disorder or symptom. An effective amount also refers to an amount sufficient to allow or facilitate diagnosis. The effective amount for a particular patient or veterinary subject may vary depending on factors such as the disorder to be treated, the general health of the patient, the method and route and dose of administration, and the severity of side effects. An effective amount may be the maximum dose or administration regimen to avoid significant side effects or toxic effects.

“Exogenous” refers to substances that are produced outside an organism, cell, or human body, depending on the context. “Endogenous” refers to substances that are produced inside a cell, organism, or human body, depending on the context.

The “sequence identity” refers to the sequence similarity between two polynucleotide sequences or between two polypeptides. When positions in two compared sequences are all occupied by identical bases or amino acid monomer subunits, for example, if a position in each of two DNA molecules is occupied by adenine, the molecules are identical at that position. The percent identity between two sequences is a function of the number of matched or homologous positions shared by the two sequences divided by the number of the compared positions×100%. For example, if 6 out of 10 positions are matched or homologous when two sequences are optimally aligned, the two sequences are of 60% identity. In general, the comparison is made when two aligned sequences to obtain the greatest percent identity. Those skilled in the art can determine the number of changed bases or amino acids indicated by the percent sequence identity.

As used herein, the expressions “cell”, “cell line”, and “cell strain” are used interchangeably, and all such designations include their progenies. Therefore, the “transformant” and “transformed cell” include primary test cells and cultures derived therefrom, regardless of the number of transfers. It should also be understood that all progenies, including mutant progenies with identical function or biological activity as screened in the originally transformed cell, may not be precisely identical in DNA content due to intentional or unintentional mutations. When referring to different designations, they will become clear through the context.

“Pharmaceutical composition” refers to a mixture comprising one or more anti-CD39 antibodies or antigen-binding fragments thereof described herein, and other components, for example, physiological/pharmaceutically acceptable carriers and excipients. The pharmaceutical composition is intended to promote the administration to an organism, so as to facilitate the absorption of the active ingredient, thereby exerting biological activity.

In the present invention, the UniProtKB/Swiss-Prot accession number of the CD39 protein is P49961.1. In the present invention, a constructed HEK293 cell overexpressing human CD39 is used as an antigen to immunize mice, so that the mice may immunoreact to generate the anti-CD39 murine antibody.

“CD39”, “CD39 antigen”, and “CD39 protein” are used interchangeably herein. CD39 is also referred to as ectonucleoside triphosphate diphosphohydrolase 1 (gene: ENTPD1; protein: NTPDasel, see www.ncbi.nlm.nih.gov/gene/953). CD39 was once referred to as ATPD enzyme or SPG64. These terms described above may be each used interchangeably. Unless otherwise indicated, the terms include any variant, subtype, and species homolog of human CD39 that is expressed naturally by cells or by cells transfected with the CD39 gene.

SK-MEL-28 is a human melanoma cell line, which naturally expresses human CD39 on the cell surface and is used for detecting the affinity of the anti-CD39 antibody for CD39 and its inhibition of the enzymatic activity of CD39. The cell is inoculated into a melanoma model established in CB17/SCID mice for detecting the in vivo anti-tumor effect of the anti-CD39 antibody.

CD4+ T cells express CD39 and CD73, which hydrolyze ATP in the surrounding environment to adenosine in a sequential reaction, thereby inhibiting CD4+ T cell proliferation. In an ATP-rich environment, an anti-CD39 antibody is added and is capable of inhibiting CD39-mediated ATP hydrolysis on the surface of CD4+ T cells, thereby relieving the inhibition of CD4+ T cell proliferation.

CD3/CD28 beads are used to activate the T cell proliferation.

MOLP-8 is a human multiple myeloma cell line, which naturally expresses human CD39 on the cell surface. The cell is inoculated into the multiple myeloma model established in CB17/SCID mice for detecting the in vivo anti-tumor effect of the anti-CD39 antibody.

The DNA sequences encoding the CDRs, variable regions, or light and heavy chains of the anti-CD39 antibody involved in the present invention can be designed based on the corresponding amino acid sequences, which is a conventional technology in the art.

The present invention is further described below with reference to examples, but these examples are not intended to limit the scope of the present invention. The experimental methods in the examples of the present invention, in which specific conditions are not specified, are generally performed under conventional conditions such as Cold Spring Harbor Antibody Technical Guide and Molecular Cloning Manual, or under conditions recommended by the manufacturer of the raw material or the goods. Reagents or materials without specific origins indicated are commercially available conventional reagents or materials.

### Example 1. Screening for Anti-CD39 Murine Antibodies

### 1.1 Construction of Cell Lines Overexpressing Human CD39

The nucleotide sequence encoding the full-length amino acid sequence of human CD39 (UniProtKB/Swiss-Prot; P49961.1) was cloned into the pLVX-IRES-Puro vector (Clontech, Catalog No.: 632183), and the resulting product was labeled as pLVX-hCD39-IRES-Puro.

The pLVX-hCD39-IRES-Puro was transfected into blank cells HEK293 and CHOK1 separately, and subcloning in a 96-well plate was performed by limiting dilution to obtain stable cell lines overexpressing human CD39, which were labeled as HEK293-hCD39 and CHOK1-hCD39 cells, respectively.

### 1.2 Hybridoma Preparation and Antibody Screening

Balb/c and SJL mice (Shanghai Slac) aged 6-8 weeks were received and housed under SPF (specific pathogen free) conditions. The HEK293-hCD39 cells described above were resuspended in phosphate buffered saline (PBS) to obtain a cell suspension at 1-2×10⁷ cells/mL. Each mouse was injected intraperitoneally with 0.5 mL of HEK293-hCD39 cell suspension at the time of immunization. The primary immunization and the second immunization were performed at an interval of 2 weeks, and the subsequent immunizations were performed at an interval of 3 weeks; a total of 3 immunizations were performed.

Except for the primary immunization, blood sampling was performed 1 week later after each booster immunization, and the titer and specificity of the anti-human CD39 antibody generated in serum were assayed by FACS. Spleen cells of mice with better serum antibody titer were selected and fused with mouse myeloma cells SP2/0 (ATCC) to prepare hybridoma cells. Specifically, another booster immunization was performed before fusion, then the mice were sacrificed after 3-5 days, and their spleen cells were collected, washed three times with DMEM medium (Gibco) and mixed with mouse myeloma cells SP2/0 (ATCC) at a ratio of 2:1-4:1 in number of viable cells, and the resulting mixture was subjected to cell fusion by the electrofusion method.

The fused cells were resuspended in DMEM (Gibco)+20% fetal bovine serum+1×HT (Invitrogen), and the suspension was seeded into a 96-well plate at 100 μL/well, and after 24 h, 100 μL of DMEM+20% fetal bovine serum+2×HAT (Invitrogen) was added, and the cell culture plate was placed in an incubator with 5% CO₂, at 37° C. When the diameter of the cell colony reached 1-2 mm (usually 10-14 days after fusion), the Acumen® eX3 cell biology high content analysis platform (Acumen) was used for determining the binding activity of cell supernatants for CHOK1-hCD39, and positive clones with relatively high mean fluorescence intensity (MFI) were selected to be expanded to a 24-well plate for expansion in DMEM+20% fetal bovine serum+1×HT. After 3 days of culturing, the supernatants in the 24-well plate were collected and subjected to second screening. The binding activity of the cell supernatants for CHOK1-hCD39 was determined by FACS (see Example 2.2.1 for the method), and the inhibition of the enzymatic activity of CD39 on the surface of the SK-MEL-28 (ATCC) cell membrane by the cell supernatants was detected (see Example 2.2.2 for the method).

According to the screening result of the 24-well plate, positive parent clones showing relatively strong inhibition of enzymatic activity were selected, subcloned in a 96-well plate by limiting dilution, and cultured in DMEM+20% fetal calf serum+1×HT under 5% CO₂ at 37° C. After 7 days of subcloning, primary screening was performed with Acumen, and positive subclones binding to CHOK1-hCD39 were selected and expanded to a 24-well plate for further culturing. A second screening was performed after 3 days, and the binding activity of the cell supernatants for CHOK1-hCD39 and their inhibition of the enzymatic activity of CD39 on the surface of the SK-MEL-28 cell membrane were detected.

According to the detecting result of the 24-well plate, preferred subclones were placed in a T75 cell culture flask for expanded culture; the expanded subclones were resuspended in DMEM+15% fetal bovine serum+10% DMSO and cryopreserved in liquid nitrogen for a long time, and thus the hybridoma cell of the present invention was obtained and the antibody derived therefrom was the anti-CD39 murine monoclonal antibody mF03 of the present invention.

### 1.3 Amino Acid Sequencing for Murine Antibody

Sequencing for the variable regions of the murine antibody mF03: mRNA of the hybridoma cell selected in Example 1 was extracted and reverse-transcribed into cDNA, then PCR was performed with a universal primer, the DNA product obtained through PCR was subjected to sequencing analysis and then translated into an amino acid sequence, and the analysis of CDR regions was performed on the variable region sequence using the Kabat scheme; the results are shown in Table 1.

TABLE 1
Amino acid sequence of anti-human CD39 murine antibody mF03

| Name | Sequence No. |
|---|---|
| Heavy chain CDR1 | SEQ ID NO: 1 |
| Heavy chain CDR2 | SEQ ID NO: 2 |
| Heavy chain CDR3 | SEQ ID NO: 3 |
| Light chain CDR1 | SEQ ID NO: 4 |
| Light chain CDR2 | SEQ ID NO: 5 |
| Light chain CDR3 | SEQ ID NO: 6 |
| Heavy chain variable region | SEQ ID NO: 7 |
| Light chain variable region | SEQ ID NO: 8 |

### Example 2. Preparation and Activity Assay of Chimeric Antibody

### 2.1 Preparation of Chimeric Antibody

cDNAs were synthesized based on the amino acid sequences of the variable regions of the heavy and light chains of the murine antibody mF03 of the present invention and inserted directionally into the expression vectors pCDNA3.4 (Invitrogen, A14697) containing signal peptide and human heavy chain IgG4 (S228P) constant region gene, and signal peptide and human light chain kappa constant region gene, respectively, then the light and heavy chains were mixed at 1:1 and transiently transfected into CHO-S cells (Thermo), and the expression supernatant was collected and the chimeric antibody expressed by CHO-S cells was purified using MabSelect PrismA (GE, 10293703) and named F03.

TABLE 2
Amino acid sequence of chimeric antibody F03

| Name of antibody | Heavy chain | Light chain |
|---|---|---|
| F03 | SEQ ID NO: 9 | SEQ ID NO: 10 |

### 2.2 Activity Assay of Chimeric Antibody

### 2.2.1 Affinity

The binding of the chimeric antibody F03 of the present invention to CD39 on the cell membrane surface was assayed by FACS. Specifically, CHOK1-hCD39 cells were collected and added to a 96-well plate at 1-3×10⁵ cells/well, the serially diluted chimeric antibody F03 (diluted with 1×PBS to 16.7 μg/mL as an initial concentration, 3-fold gradient dilution) was added, and the mixture was incubated at 4° C. for 30 min; after washing with PBS, an FITC-labeled goat anti-human IgG Fc secondary antibody (Jackson Immunoresearch, 109095008) diluted at 1:200 was added, and the resulting mixture was incubated at 4° C. for 30 min; after washing with PBS, the cell fluorescence intensity was detected using an FACS instrument (Beckman). Data were processed by GraphPad software.

The antibody 31895 was prepared with reference to the antibody sequences and method described in U.S. Pat. No. US20190062448A1 and used in the positive control group for a control test. The antibody 31895 is also referred to as TTX-030 and is currently used in clinical trials by the company Tizona.

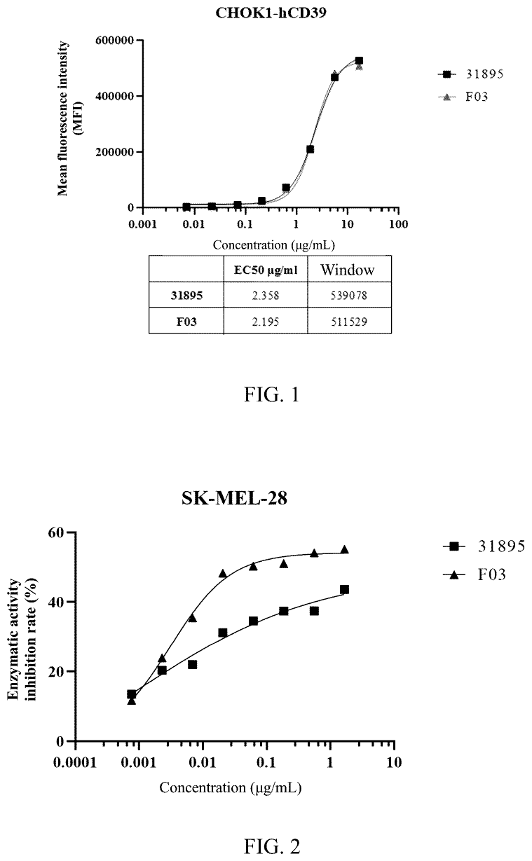

The results are shown in FIG. 1. The chimeric antibody F03 of the present invention and the control antibody 31895 have a similar binding activity for human CD39 on the cell membrane surface.

### 2.2.2 Inhibition of Enzymatic Activity of CD39 on the Cell Surface

SK-MEL-28 cells were seeded into wells of a 96-well plate at 50 μL/well (1.2-2.5×10⁵/mL), the chimeric antibody F03 serially diluted in EMEM+10% FBS (ATCC) medium (at an initial concentration of 1.67 μg/mL, 3-fold gradient dilution) was added, and the mixture was incubated overnight at 37° C.; the supernatant was discarded, the cells were washed once with PBS, 40 μM ATP (Sigma, A7699) was added, and the resulting mixture was incubated at 37° C. for 1 h; after centrifugation at 300 g for 5 min, the supernatant was transferred to a 96-well removable opaque cell plate (Thermo, 463201), and the CTG solution (CellTiter-Glo® Luminescent Cell Viability Assay, Promega G7571) was added at a ratio of 1:1; after the mixture was being away from light for 10 min, the chemiluminescence value was read with a multifunctional microplate reader (Molecular Devices). Meanwhile, ATP control wells (with SK-MEL-28 cells and antibody not added, ATP with an equal amount of the experimental group added, and others same as the experimental group), cell+ATP control wells (with SK-MEL-28 cells and ATP added, antibody not added, and others same as the experimental group), and positive control wells (with antibody 31895 in Example 2.2.1 as the positive control antibody, and others same as the experimental group) were provided. Calculation formula for enzymatic activity inhibition rate: ((cell+ATP+antibody)−(cell+ATP))/(ATP−(cell+ATP))×100%.

The results are shown in FIG. 2. The chimeric antibody F03 of the present invention can well inhibit the enzymatic activity of CD39 on the cell surface for hydrolyzing ATP, with inhibitory effect better than that of the positive control antibody 31895.

### Example 3. Preparation and Detection of Humanized Antibodies

### 3.1 Sequence Design for Humanization and Antibody Preparation

The humanization was performed on the basis of the heavy chain variable region and the light chain variable region of the murine antibody mF03 of the present invention. Six CDRs of the heavy and light chains of the murine antibody were grafted into a humanization template having a relatively high similarity to murine FR regions. The sequences of germline genes with the highest homology to the heavy chain variable region and the light chain variable region of mF03 were selected as the humanization templates by sequence alignment (NCBI-Igblast), wherein the heavy chain variable region template was human germline heavy chain IGHV1-2%*02 and the light chain variable region template was human germline light chain IGKV1-33*01.

The CDR-grafted antibody was subjected to homology modeling to predict the key amino acids in the FR regions of the murine antibody which may determine the antibody structure, and amino acids of a certain humanization template of the FR region were back mutated to the murine amino acids of the corresponding positions.

Sequencing of the obtained humanized antibodies: cDNAs were synthesized based on the amino acid sequences of the heavy chain variable region and light chain variable region of the humanized antibody and inserted into the pcDNA3.4 expression vectors containing signal peptide and human IgG4 (S228P) heavy chain constant region gene, and signal peptide and human light chain kappa constant region gene, respectively, thus obtaining expression plasmids of the full-length antibody. The heavy chain and light chain expression plasmids were co-transfected into CHO-S cells, and after culturing, the supernatants were harvested and purified using MabSelect PrismA to obtain the humanized antibodies of the present invention.

TABLE 3
Sequences of humanized antibodies

| | Heavy chain | Light chain | Heavy chain | Light chain |
|---|---|---|---|---|
| | variable | variable | constant | constant |
| Antibody | region | region | region | region |
| F03-6 | SEQ ID NO: 11 | SEQ ID NO: 16 | SEQ ID NO: 18 | SEQ ID NO: 19 |
| F03-7 | SEQ ID NO: 12 | SEQ ID NO: 16 | | |
| F03-8 | SEQ ID NO: 13 | SEQ ID NO: 16 | | |
| F03-9 | SEQ ID NO: 14 | SEQ ID NO: 16 | | |
| F03-10 | SEQ ID NO: 15 | SEQ ID NO: 16 | | |
| F03-11 | SEQ ID NO: 11 | SEQ ID NO: 17 | | |
| F03-12 | SEQ ID NO: 12 | SEQ ID NO: 17 | | |
| F03-13 | SEQ ID NO: 13 | SEQ ID NO: 17 | | |
| F03-14 | SEQ ID NO: 14 | SEQ ID NO: 17 | | |
| F03-15 | SEQ ID NO: 15 | SEQ ID NO: 17 | | |

### 3.2 Detection of Humanized Antibodies

### 3.2.1 Determination of Affinity and Inhibition of Enzymatic Activity for ATP

The humanized antibodies of the present invention were tested, with reference to methods in Examples 2.2.1 and 2.2.2, for affinity for SK-MEL-28 cells and inhibition of the enzymatic activity of CD39 on the surface of SK-MEL-28 cells for hydrolyzing ATP.

The results are shown in Table 4. The affinity of F03-6 to F03-15 for SK-MEL-28 cells was comparable to that of the chimeric antibody F03; the inhibition of the enzymatic activity of CD39 on the cell surface for hydrolyzing ATP by F03-6 to F03-15 was comparable to that of the chimeric antibody F03.

TABLE 4
Cell affinity and enzymatic activity
inhibition of humanized antibodies

SK-MEL-28 cells

| Antibody | Affinity | Enzymatic activity |
|---|---|---|
| No. | (EC50 nM) | inhibition (EC50 nM) |
| F03 | 2.07 | 0.09 |
| F03-6 | 1.27 | 0.09 |
| F03-7 | 0.89 | 0.15 |
| F03-8 | 1.00 | 0.06 |
| F03-9 | 1.01 | 0.04 |
| F03-10 | 1.48 | 0.02 |
| F03-11 | 0.94 | 0.03 |
| F03-12 | 1.12 | 0.06 |
| F03-13 | 1.00 | 0.09 |
| F03-14 | 0.79 | 0.05 |
| F03-15 | 1.11 | 0.02 |

### 3.2.2 Assay for Inhibition of CD4+ T Cell Proliferation

Frozen human peripheral blood mononuclear cells (PBMCs) were thawed and cultured overnight in 1640+10% FBS (Gibco) medium, and CD4+ T cells were isolated from the PBMCs using the miltenyi biotec (cat #130-096-533) kit; the isolated CD4+ T cells were stained with carboxyfluorescein diacetate succinimidyl ester (CFSE) to obtain the CD4+ T-CFSE. The CD4+T-CFSE was mixed with CD3/CD28 beads (Gibco, 11161D) at 5:1, and then the cells were plated at 1.25×10⁵ cells/well (50 μL/well); 50 μL of serially diluted test antibody was added, and the mixture was incubated in an incubator for 30-60 min; finally, 500-1000 μM ATP was added at 50 μL/well, the resulting mixture was incubated in a cell incubator for 3-5 days, and the CD4+ T cell proliferation was detected by a flow cytometer.

The I-394 antibody was prepared with reference to the antibody sequence and method described in WO2019096900A1 and used in the positive control group; the control groups set included: 1) an activated CD4+ T-CFSE control group, in which antibody and ATP were not added, and others were the same as the experimental group, representing that T cells can be activated under a normal state; 2) an activated CD4+ T-CFSE+ATP control group, in which antibody was not added, and others were the same as the experimental group, representing inhibition of proliferation of T cells after addition of ATP; the experimental group was intended to investigate whether the test antibody had the effect of reversing the inhibition of T cell proliferation.

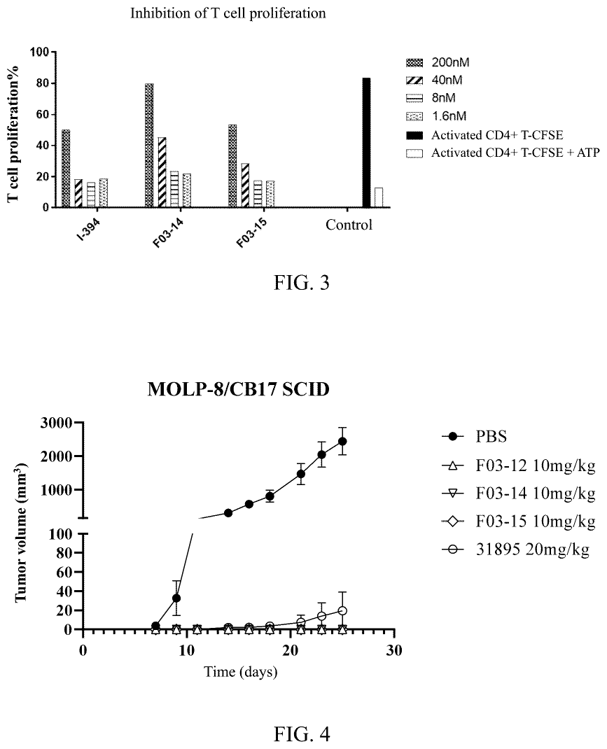

The results are shown in FIG. 3. The humanized antibodies of the present invention can well reverse the inhibition of CD4+ T cell proliferation, and the effect of F03-14 and F03-15 is better than that of the positive control antibody I-394.

### 3.2.3 Affinity Assay by Surface Plasmon Resonance

The binding kinetics between humanized antibodies and a recombinant antigen were tested using Biacore 8K (GE, biomolecule interaction analyzer). Specifically, an anti-human IgG Fc antibody was directly immobilized on a biosensor chip to capture a test antibody. The antigen hCD39-ECD-His (ACRO, CD9-H52H4) was used as the mobile phase, and 7 concentration gradients (at an initial concentration of 200 nM, 2-fold gradient dilution) were used. The association rate constant ka (M⁻¹ s⁻¹) and dissociation rate constant kd (s⁻¹) were determined. Then, based on the formula KD=kd/ka and the kinetic rate constants, the equilibrium dissociation constant KD (M) of the reaction between the antibody and the relevant target protein was calculated. The results are shown in Table 5. The humanized antibodies of the present invention maintained high affinity of the chimeric antibody F03 for the target antigen.

TABLE 5
Affinity of humanized antibodies

| Antibody No. | ka (1/Ms) | kd (1/s) | KD (M) |
|---|---|---|---|
| F03 | 9.43E+04 | 2.52E−04 | 2.67E−09 |
| F03-11 | 8.01E+04 | 3.36E−04 | 4.20E−09 |
| F03-12 | 8.99E+04 | 3.31E−04 | 3.68E−09 |
| F03-14 | 6.98E+04 | 3.10E−04 | 4.43E−09 |
| F03-15 | 5.28E+04 | 1.76E−04 | 3.33E−09 |

### 3.2.4 In Vivo Activity in Resisting Multiple Myeloma

CB17/SCID mice (Shanghai Slac, aged 6-8 weeks) were randomized into groups of 5; on day 0, 5×10⁶ MOLP-8 cells (multiple myeloma cells, DSMZ) were inoculated into the right abdomen of each mouse; on day 1, the administration was started (including antibodies F03-12, F03-14, and F03-15 of the present invention, and the control antibody 31895, the blank control group being injected with PBS at a volume equivalent to that of the substances injected in the experimental groups); intravenous injection and intraperitoneal injection were alternately performed, the administration was performed twice a week for 3 weeks continuously, the dose of the antibodies (F03-12, F03-14, and F03-15) injected into mice in the experimental groups was 10 mg/kg, and the dose of the antibody injected into mice in the 31895 control group was 20 mg/kg. Tumor growth was monitored over time by measuring the length and width of the tumor with a vernier caliper, and the tumor volume was calculated by the formula TV=(length×width²)/2; the average of the tumor volume of each group of mice was used to plot tumor growth curves.

The results are shown in FIG. 4. As of day 25, the humanized antibodies and the positive control 31895 all demonstrated good inhibitory effect on tumor growth, and humanized antibodies F03-12, F03-14, and F03-15 showed better effect than the positive control 31895.

### 3.2.5 In Vivo Activity in Resisting Melanoma

CB17/SCID mice (Shanghai Slac, aged 6-8 weeks) were randomized into groups of 5; on day 0, 1×10⁷ SK-MEL-28 cells (melanoma cells, ATCC) were inoculated into the right abdomen of each mouse, wherein the cells were mixed with an equal volume of matrigel (Corning) in advance; on day 1, the administration was started (the antibody F03-14 of the present invention, the blank control group being injected with PBS at a volume equivalent to that of the substance injected in the experimental group); intravenous injection and intraperitoneal injection were alternately performed, the administration was performed twice a week for 3 weeks continuously, and the dose of the antibody injected per mouse was 10 mg/kg. Tumor growth was monitored over time by measuring the length and width of the tumor with a vernier caliper, and the tumor volume was calculated by the formula TV=(length×width²)/2; the average of the tumor volume of each group of mice was used to plot tumor growth curves.

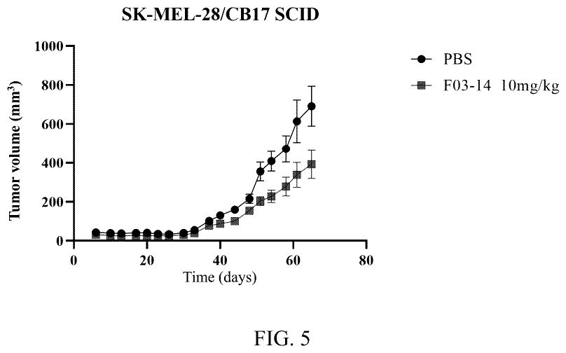

The results are shown in FIG. 5. F03-14 demonstrated better inhibitory effect on tumor growth compared with the blank control.

### Sequences:

SEQ ID NO: 1:
NYWMY
SEQ ID NO: 2:
RIAPSSGATKYNEKFKN
SEQ ID NO: 3:
SGIYYDYDWFFDV
SEQ ID NO: 4:
KASQDINKYIG
SEQ ID NO: 5:
YTSTLQP
SEQ ID NO: 6:
LQYHALLFT
SEQ ID NO: 7:
QVQLQQPGAELVKPGASVKLSCKTSGYTFTNYWMYWVKHRPGRGLE
WIGRIAPSSGATKYNEKFKNKATLTVDKPSSTAYMQLSSLTSEDSAVYYC
ARSGIYYDYDWFFDVSGTGTTVTVSS
SEQ ID NO: 8:
DIQMTQSPSSLSASLGGKVTITCKASQDINKYIGWYQHAPGKGPRLLIHY
TSTLQPGIPSRFSGSGSGRDYSLSISNLEPEDIATYYCLQYHALLFTFGS
GTKLELK
SEQ ID NO: 9:
QVQLQQPGAELVKPGASVKLSCKTSGYTFTNYWMYWVKHRPGRGLE
WIGRIAPSSGATKYNEKFKNKATLTVDKPSSTAYMQLSSLTSEDSAVYYC
ARSGIYYDYDWFFDVSGTGTTVTVSSASTKGPSVFPLAPCSRSTSESTAA
LGCLVKDYFPEPVTVSWNSGALTSGVHTFPAVLQSSGLYSLSSVVTVPSS
SLGTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSLS
LGK
SEQ ID NO: 10:
DIQMTQSPSSLSASLGGKVTITCKASQDINKYIGWYQHAPGKGPRLLIHY
TSTLQPGIPSRFSGSGSGRDYSLSISNLEPEDIATYYCLQYHALLFTFGS
GTKLELKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID NO: 11:
QVQLVQSGAEVKKPGASVKVSCKASGYTFTNYWMYWVRQAPGQGLE
WMGRIAPSSGATKYNEKFKNRVTMTRDTSISTAYMELSRLRSDDTAVYYC
ARSGIYYDYDWFFDVWGQGTTVTVSS
SEQ ID NO: 12:
QVQLVQSGAEVKKPGASVKVSCKASGYTFTNYWMYWVRQAPGQGL
EWMGRIAPSSGATKYNEKFKNRVTLTVDTSISTAYMELSRLRSDDTAVYY
CARSGIYYDYDWFFDVWGQGTTVTVSS
SEQ ID NO: 13:
QVQLVQSGAEVKKPGASVKVSCKASGYTFTNYWMYWVRQAPGQGL
EWIGRIAPSSGATKYNEKFKNRATLTVDTSISTAYMELSRLRSDDTAVYY
CARSGIYYDYDWFFDVWGQGTTVTVSS
SEQ ID NO: 14:
QVQLVQSGAEVKKPGASVKVSCKASGYTFTNYWMYWVRQRPGRGLE
WIGRIAPSSGATKYNEKFKNRATLTVDTSISTAYMELSRLRSDDTAVYYC
ARSGIYYDYDWFFDVWGQGTTVTVSS
SEQ ID NO: 15:
QVQLVQSGAEVKKPGASVKVSCKASGYTFTNYWMYWVRQRPGRGLE
WIGRIAPSSGATKYNEKFKNRATLTVDTSISTAYMELSRLRSDDTAVYYC
ARSGIYYDYDWFFDVSGQGTTVTVSS
SEQ ID NO: 16:
DIQMTQSPSSLSASVGDRVTITCKASQDINKYIGWYQQKPGKAPKLLIH
YTSTLQPGVPSRFSGSGSGTDFTFTISSLQPEDIATYYCLQYHALLFTFG
QGTKLEIK
SEQ ID NO: 17:
DIQMTQSPSSLSASVGDRVTITCKASQDINKYIGWYQQKPGKAPKLLIH
YTSTLQPGVPSRFSGSGSGRDYTFTISSLQPEDIATYYCLQYHALLFTFG
QGTKLEIK
SEQ ID NO: 18:
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRVE
SKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK
SEQ ID NO: 19:
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSSPV
TKSFNRGEC

1. An anti-CD39 antibody or an antigen-binding fragment thereof, comprising:
a heavy chain variable region comprising at least 1 HCDR as follows:
HCDR1, the amino acid sequence of the HCDR1 being set forth in SEQ ID NO: 1 or the HCDR1 comprising the amino acid sequence set forth in SEQ ID NO: 1;
HCDR2, the amino acid sequence of the HCDR2 being set forth in SEQ ID NO: 2 or the HCDR2 comprising the amino acid sequence set forth in SEQ ID NO: 2;
HCDR3, the amino acid sequence of the HCDR3 being set forth in SEQ ID NO: 3 or the HCDR3 comprising the amino acid sequence set forth in SEQ ID NO: 3;
and/or
a light chain variable region comprising at least 1 LCDR as follows:
LCDR1, the amino acid sequence of the LCDR1 being set forth in SEQ ID NO: 4 or the LCDR1 comprising the amino acid sequence set forth in SEQ ID NO: 4;
LCDR2, the amino acid sequence of the LCDR2 being set forth in SEQ ID NO: 5 or the LCDR2 comprising the amino acid sequence set forth in SEQ ID NO: 5;
LCDR3, the amino acid sequence of the LCDR3 being set forth in SEQ ID NO: 6 or the LCDR3 comprising the amino acid sequence set forth in SEQ ID NO: 6;
wherein preferably, the heavy chain variable region comprises the HCDR1, the HCDR2, and the HCDR3 set forth in SEQ ID NO: 1, SEQ ID NO: 2, and SEQ ID NO: 3, respectively; and/or
the light chain variable region comprises the LCDR1, the LCDR2, and the LCDR3 set forth in SEQ ID NO: 4, SEQ ID NO: 5, and SEQ ID NO: 6, respectively.

2. The anti-CD39 antibody or the antigen-binding fragment thereof according to claim 1, further comprising a heavy chain FR region of a murine IgG1, IgG2, IgG3 or IgG4 or a variant thereof, and/or further comprising a light chain FR region of a murine κ or λ chain or a variant thereof;
wherein preferably, the amino acid sequence of the heavy chain variable region is set forth in SEQ ID NO: 7 or has at least 90%, at least 95%, at least 96%, at least 97%, at least 98%, or at least 99% sequence identity to SEQ ID NO: 7; and/or the amino acid sequence of the light chain variable region is set forth in SEQ ID NO: 8 or has at least 90%, at least 95%, at least 96%, at least 97%, at least 98%, or at least 99% sequence identity to SEQ ID NO: 8.

3. The anti-CD39 antibody or the antigen-binding fragment thereof according to claim 1, being a murine antibody or an antigen-binding fragment thereof, wherein the anti-CD39 antibody or the antigen-binding fragment thereof further comprises a heavy chain constant region of the murine IgG1, IgG2, IgG3 or IgG4 or the variant thereof and/or further comprises a light chain constant region of the murine κ or λ chain or the variant thereof.

4. The anti-CD39 antibody or the antigen-binding fragment thereof according to claim 1, being a chimeric antibody or an antigen-binding fragment thereof, wherein the anti-CD39 antibody or the antigen-binding fragment thereof further comprises a heavy chain constant region of a human IgG1, IgG2, IgG3 or IgG4 or a variant thereof, preferably a heavy chain constant region of a human IgG4 or a variant thereof, and/or further comprises a light chain constant region of a human κ or λ chain or a variant thereof, preferably a light chain constant region of a human κ or a variant thereof;
preferably, the heavy chain amino acid sequence of the chimeric antibody is set forth in SEQ ID NO: 9 or has at least 90%, at least 95%, at least 96%, at least 97%, at least 98%, or at least 99% sequence identity to SEQ ID NO: 9; and/or the light chain amino acid sequence of the antibody is set forth in SEQ ID NO: 10 or has at least 90%, at least 95%, at least 96%, at least 97%, at least 98%, or at least 99% sequence identity to SEQ ID NO: 10.

5. The anti-CD39 antibody or the antigen-binding fragment thereof according to claim 1, being a humanized antibody or an antigen-binding fragment thereof, wherein the anti-CD39 antibody or the antigen-binding fragment thereof further comprises a heavy chain FR region of a human IgG1, IgG2, IgG3 or IgG4 or a variant thereof, preferably a FR region of human germline heavy chain IGHV1-2*02 or a variant thereof, and/or further comprises a light chain FR region of a human κ or λ chain or a variant thereof, preferably a FR region of human germline light chain IGKV1-33*01 or a variant thereof;
preferably, the amino acid sequence of the heavy chain variable region is set forth in SEQ ID NO: 11, 12, 13, 14, or 15 or has at least 90%, at least 95%, at least 96%, at least 97%, at least 98%, or at least 99% sequence identity to SEQ ID NO: 11, 12, 13, 14, or 15; and/or the amino acid sequence of the light chain variable region is set forth in SEQ ID NO: 16 or 17 or has at least 90%, at least 95%, at least 96%, at least 97%, at least 98%, or at least 99% sequence identity to SEQ ID NO: 16 or 17;
further preferably, the amino acid sequence of the heavy chain variable region of the anti-CD39 antibody or the antigen-binding fragment thereof is set forth in SEQ ID NO: 11 or has at least 90%, at least 95%, at least 96%, at least 97%, at least 98%, or at least 99% sequence identity to SEQ ID NO: 11, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16; or
the amino acid sequence of the heavy chain variable region of the anti-CD39 antibody or the antigen-binding fragment thereof is set forth in SEQ ID NO: 12 or has at least 90%, at least 95%, at least 96%, at least 97%, at least 98%, or at least 99% sequence identity to SEQ ID NO: 12, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16; or
the amino acid sequence of the heavy chain variable region of the anti-CD39 antibody or the antigen-binding fragment thereof is set forth in SEQ ID NO: 13 or has at least 90%, at least 95%, at least 96%, at least 97%, at least 98%, or at least 99% sequence identity to SEQ ID NO: 13, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16; or
the amino acid sequence of the heavy chain variable region of the anti-CD39 antibody or the antigen-binding fragment thereof is set forth in SEQ ID NO: 14 or has at least 90%, at least 95%, at least 96%, at least 97%, at least 98%, or at least 99% sequence identity to SEQ ID NO: 14, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16; or
the amino acid sequence of the heavy chain variable region of the anti-CD39 antibody or the antigen-binding fragment thereof is set forth in SEQ ID NO: 15 or has at least 90%, at least 95%, at least 96%, at least 97%, at least 98%, or at least 99% sequence identity to SEQ ID NO: 15, and the amino acid sequence of the light chain variable region of the anti-CD39 antibody or the antigen-binding fragment thereof is set forth in SEQ ID NO: 16 or has at least 90%, at least 95%, at least 96%, at least 97%, at least 98%, or at least 99% sequence identity to SEQ ID NO: 16; or
the amino acid sequence of the heavy chain variable region of the anti-CD39 antibody or the antigen-binding fragment thereof is set forth in SEQ ID NO: 11 or has at least 90%, at least 95%, at least 96%, at least 97%, at least 98%, or at least 99% sequence identity to SEQ ID NO: 11, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17; or
the amino acid sequence of the heavy chain variable region of the anti-CD39 antibody or the antigen-binding fragment thereof is set forth in SEQ ID NO: 12 or has at least 90%, at least 95%, at least 96%, at least 97%, at least 98%, or at least 99% sequence identity to SEQ ID NO: 12, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17; or
the amino acid sequence of the heavy chain variable region of the anti-CD39 antibody or the antigen-binding fragment thereof is set forth in SEQ ID NO: 13 or has at least 90%, at least 95%, at least 96%, at least 97%, at least 98%, or at least 99% sequence identity to SEQ ID NO: 13, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17; or
the amino acid sequence of the heavy chain variable region of the anti-CD39 antibody or the antigen-binding fragment thereof is set forth in SEQ ID NO: 14 or has at least 90%, at least 95%, at least 96%, at least 97%, at least 98%, or at least 99% sequence identity to SEQ ID NO: 14, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17; or
the amino acid sequence of the heavy chain variable region of the anti-CD39 antibody or the antigen-binding fragment thereof is set forth in SEQ ID NO: 15 or has at least 90%, at least 95%, at least 96%, at least 97%, at least 98%, or at least 99% sequence identity to SEQ ID NO: 15, and the amino acid sequence of the light chain variable region of the anti-CD39 antibody or the antigen-binding fragment thereof is set forth in SEQ ID NO: 17 or has at least 90%, at least 95%, at least 96%, at least 97%, at least 98%, or at least 99% sequence identity to SEQ ID NO: 17.

6. The anti-CD39 antibody or the antigen-binding fragment thereof according to claim 5, further comprising a heavy chain constant region of a human IgG1, IgG2, IgG3 or IgG4 or a variant thereof, preferably a heavy chain constant region of a human IgG4 or a variant thereof, and/or further comprising a light chain constant region of a human κ or λ chain or a variant thereof, preferably a light chain constant region of a human κ or a variant thereof;
wherein preferably, the amino acid sequence of the heavy chain constant region is set forth in SEQ ID NO: 18 or has at least 90%, at least 95%, at least 96%, at least 97%, at least 98%, or at least 99% sequence identity to SEQ ID NO: 18, and/or the amino acid sequence of the light chain constant region is set forth in SEQ ID NO: 19 or has at least 90%, at least 95%, at least 96%, at least 97%, at least 98%, or at least 99% sequence identity to SEQ ID NO: 19.

7. The anti-CD39 antibody or the antigen-binding fragment thereof according to claim 1, wherein the antigen-binding fragment is selected from Fab, Fv, scFv, Fab′, or F(ab′)².

8. A biomaterial, being
(1) a DNA molecule encoding the antibody or the antigen-binding fragment thereof according to claim 1;
(2) an expression vector comprising the DNA molecule according to (1);
(3) a host cell or a culture thereof comprising the DNA molecule according to (1) or the expression vector according to (2), wherein the host cell is preferably a human embryonic kidney 293 cell or a Chinese hamster ovary cell.

9. A method for producing an anti-CD39 antibody or an antigen-binding fragment thereof, comprising: culturing the host cell according to claim 8, wherein preferably, the method further comprises isolating an antibody from the obtained culture and purifying the antibody.

10. A pharmaceutical composition, comprising the anti-CD39 antibody or the antigen-binding fragment thereof according to claim 1 and a pharmaceutically acceptable excipient, diluent, or carrier.

11. A detection or diagnostic kit, comprising the anti-CD39 antibody or the antigen-binding fragment thereof according to claim 1.

12. (canceled)

13. A method for treating or preventing a CD39-mediated disease or disorder, comprising administering to a patient in need thereof a therapeutically effective amount of the anti-CD39 antibody or the antigen-binding fragment thereof according to claim 1, wherein the CD39-mediated disease or disorder is preferably a tumor expressing CD39, more preferably multiple myeloma, colorectal cancer, head and neck cancer, pancreatic cancer, bladder cancer, brain cancer, breast cancer, gastric cancer, hepatocellular cancer, lung cancer, leukemia, lymphoma, melanoma, ovarian cancer, prostate cancer, pituitary cancer, esophageal cancer, soft tissue sarcoma, peritoneal cancer, glioblastoma, cervical cancer, uterine cancer, salivary gland cancer, kidney cancer, vulvar cancer, thyroid cancer, testicular cancer, cholangiocarcinoma, or gallbladder cancer.

14. A method for detection, diagnosis, or prognosis of CD39 or a CD39-mediated disease or disorder, comprising: contacting a sample of a patient in need thereof with the anti-CD39 antibody or the antigen-binding fragment thereof according to claim 1 such that the anti-CD39 antibody or the antigen-binding fragment thereof binds to CD39, and detecting the binding that reflects the CD39 expression in the sample, wherein the CD39-mediated disease or disorder is preferably a tumor expressing CD39, more preferably multiple myeloma, colorectal cancer, head and neck cancer, pancreatic cancer, bladder cancer, brain cancer, breast cancer, gastric cancer, hepatocellular cancer, lung cancer, leukemia, lymphoma, melanoma, ovarian cancer, prostate cancer, pituitary cancer, esophageal cancer, soft tissue sarcoma, peritoneal cancer, glioblastoma, cervical cancer, uterine cancer, salivary gland cancer, kidney cancer, vulvar cancer, thyroid cancer, testicular cancer, cholangiocarcinoma, or gallbladder cancer.

15. A pharmaceutical composition, comprising the anti-CD39 antibody or the antigen-binding fragment thereof according to claim 4 and a pharmaceutically acceptable excipient, diluent, or carrier.

16. A pharmaceutical composition, comprising the anti-CD39 antibody or the antigen-binding fragment thereof according to claim 5 and a pharmaceutically acceptable excipient, diluent, or carrier.

17. A pharmaceutical composition, comprising the anti-CD39 antibody or the antigen-binding fragment thereof according to claim 6 and a pharmaceutically acceptable excipient, diluent, or carrier.

18. A method for treating or preventing a CD39-mediated disease or disorder, comprising administering to a patient in need thereof a therapeutically effective amount of the biomaterial according to claim 8, wherein the CD39-mediated disease or disorder is preferably a tumor expressing CD39, more preferably multiple myeloma, colorectal cancer, head and neck cancer, pancreatic cancer, bladder cancer, brain cancer, breast cancer, gastric cancer, hepatocellular cancer, lung cancer, leukemia, lymphoma, melanoma, ovarian cancer, prostate cancer, pituitary cancer, esophageal cancer, soft tissue sarcoma, peritoneal cancer, glioblastoma, cervical cancer, uterine cancer, salivary gland cancer, kidney cancer, vulvar cancer, thyroid cancer, testicular cancer, cholangiocarcinoma, or gallbladder cancer.

19. A method for treating or preventing a CD39-mediated disease or disorder, comprising administering to a patient in need thereof a therapeutically effective amount of the pharmaceutical composition according to claim 10, wherein the CD39-mediated disease or disorder is preferably a tumor expressing CD39, more preferably multiple myeloma, colorectal cancer, head and neck cancer, pancreatic cancer, bladder cancer, brain cancer, breast cancer, gastric cancer, hepatocellular cancer, lung cancer, leukemia, lymphoma, melanoma, ovarian cancer, prostate cancer, pituitary cancer, esophageal cancer, soft tissue sarcoma, peritoneal cancer, glioblastoma, cervical cancer, uterine cancer, salivary gland cancer, kidney cancer, vulvar cancer, thyroid cancer, testicular cancer, cholangiocarcinoma, or gallbladder cancer.

20. A method for detection, diagnosis, or prognosis of CD39 or a CD39-mediated disease or disorder, comprising: contacting a sample of a patient in need thereof with the biomaterial according to claim 8 such that the anti-CD39 antibody or the antigen-binding fragment thereof binds to CD39, and detecting the binding that reflects the CD39 expression in the sample, wherein the CD39-mediated disease or disorder is preferably a tumor expressing CD39, more preferably multiple myeloma, colorectal cancer, head and neck cancer, pancreatic cancer, bladder cancer, brain cancer, breast cancer, gastric cancer, hepatocellular cancer, lung cancer, leukemia, lymphoma, melanoma, ovarian cancer, prostate cancer, pituitary cancer, esophageal cancer, soft tissue sarcoma, peritoneal cancer, glioblastoma, cervical cancer, uterine cancer, salivary gland cancer, kidney cancer, vulvar cancer, thyroid cancer, testicular cancer, cholangiocarcinoma, or gallbladder cancer.

21. A method for detection, diagnosis, or prognosis of CD39 or a CD39-mediated disease or disorder, comprising: contacting a sample of a patient in need thereof with the pharmaceutical composition according to claim 10 such that the anti-CD39 antibody or the antigen-binding fragment thereof binds to CD39, and detecting the binding that reflects the CD39 expression in the sample, wherein the CD39-mediated disease or disorder is preferably a tumor expressing CD39, more preferably multiple myeloma, colorectal cancer, head and neck cancer, pancreatic cancer, bladder cancer, brain cancer, breast cancer, gastric cancer, hepatocellular cancer, lung cancer, leukemia, lymphoma, melanoma, ovarian cancer, prostate cancer, pituitary cancer, esophageal cancer, soft tissue sarcoma, peritoneal cancer, glioblastoma, cervical cancer, uterine cancer, salivary gland cancer, kidney cancer, vulvar cancer, thyroid cancer, testicular cancer, cholangiocarcinoma, or gallbladder cancer.


SEQUENCE LISTING (19 sequences)

SEQ ID NO: 1 - protein, 5 aa, synthetic construct
NYWMY

SEQ ID NO: 2 - protein, 17 aa, synthetic construct
RIAPSSGATKYNEKFKN

SEQ ID NO: 3 - protein, 13 aa, synthetic construct
SGIYYDYDWFFDV

SEQ ID NO: 4 - protein, 11 aa, synthetic construct
KASQDINKYIG

SEQ ID NO: 5 - protein, 7 aa, synthetic construct
YTSTLQP

SEQ ID NO: 6 - protein, 9 aa, synthetic construct
LQYHALLFT

SEQ ID NO: 7 - protein, 122 aa, synthetic construct
QVQLQQPGAELVKPGASVKLSCKTSGYTFTNYWMYWVKHRPGRGLEWIGRIAPSSGATKYNEKFKNKATLTVDKPSSTAYMQLSSLTSEDSAVYYCARSGIYYDYDWFFDVSGTGTTVTVSS

SEQ ID NO: 8 - protein, 107 aa, synthetic construct
DIQMTQSPSSLSASLGGKVTITCKASQDINKYIGWYQHAPGKGPRLLIHYTSTLQPGIPSRFSGSGSGRDYSLSISNLEPEDIATYYCLQYHALLFTFGSGTKLELK

SEQ ID NO: 9 - protein, 449 aa, synthetic construct
QVQLQQPGAELVKPGASVKLSCKTSGYTFTNYWMYWVKHRPGRGLEWIGRIAPSSGATKYNEKFKNKATLTVDKPSSTAYMQLSSLTSEDSAVYYCARSGIYYDYDWFFDVSGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 214 aa, synthetic construct
DIQMTQSPSSLSASLGGKVTITCKASQDINKYIGWYQHAPGKGPRLLIHYTSTLQPGIPSRFSGSGSGRDYSLSISNLEPEDIATYYCLQYHALLFTFGSGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 122 aa, synthetic construct
QVQLVQSGAEVKKPGASVKVSCKASGYTFTNYWMYWVRQAPGQGLEWMGRIAPSSGATKYNEKFKNRVTMTRDTSISTAYMELSRLRSDDTAVYYCARSGIYYDYDWFFDVWGQGTTVTVSS

SEQ ID NO: 12 - protein, 122 aa, synthetic construct
QVQLVQSGAEVKKPGASVKVSCKASGYTFTNYWMYWVRQAPGQGLEWMGRIAPSSGATKYNEKFKNRVTLTVDTSISTAYMELSRLRSDDTAVYYCARSGIYYDYDWFFDVWGQGTTVTVSS

SEQ ID NO: 13 - protein, 122 aa, synthetic construct
QVQLVQSGAEVKKPGASVKVSCKASGYTFTNYWMYWVRQAPGQGLEWIGRIAPSSGATKYNEKFKNRATLTVDTSISTAYMELSRLRSDDTAVYYCARSGIYYDYDWFFDVWGQGTTVTVSS

SEQ ID NO: 14 - protein, 122 aa, synthetic construct
QVQLVQSGAEVKKPGASVKVSCKASGYTFTNYWMYWVRQRPGRGLEWIGRIAPSSGATKYNEKFKNRATLTVDTSISTAYMELSRLRSDDTAVYYCARSGIYYDYDWFFDVWGQGTTVTVSS

SEQ ID NO: 15 - protein, 122 aa, synthetic construct
QVQLVQSGAEVKKPGASVKVSCKASGYTFTNYWMYWVRQRPGRGLEWIGRIAPSSGATKYNEKFKNRATLTVDTSISTAYMELSRLRSDDTAVYYCARSGIYYDYDWFFDVSGQGTTVTVSS

SEQ ID NO: 16 - protein, 107 aa, synthetic construct
DIQMTQSPSSLSASVGDRVTITCKASQDINKYIGWYQQKPGKAPKLLIHYTSTLQPGVPSRFSGSGSGTDFTFTISSLQPEDIATYYCLQYHALLFTFGQGTKLEIK

SEQ ID NO: 17 - protein, 107 aa, synthetic construct
DIQMTQSPSSLSASVGDRVTITCKASQDINKYIGWYQQKPGKAPKLLIHYTSTLQPGVPSRFSGSGSGRDYTFTISSLQPEDIATYYCLQYHALLFTFGQGTKLEIK

SEQ ID NO: 18 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 107 aa, synthetic construct [sequence omitted]